(19) United States
(12) Patent Application Publication
(10) Pub. No.: US 2006/0200211 A1
(43) Pub. Date: Sep. 7, 2006

(54) Cosmetic light therapy system

(75) Inventor: Timothy Lin, Taipei (TW)

Correspondence Address: BRUCE H. TROXELL, SUITE 1404, 5205 LEESBURG PIKE, FALLS CHURCH, VA, 22041, US

(73) Assignee: Biotek Technology Corp.

(21) Appl. No.: 11/124,230

(22) Filed: May 9, 2005

(30) Foreign Application Priority Data
Mar. 4, 2005 (TW) 94203352

(51) Int. Cl. A61N 5/06

(52) U.S. Cl. 607/88

(57) ABSTRACT

The present invention discloses an improve cosmetic light therapy system having a light source of cosmetic and therapeutic capability, that can generate coherent light or noncoherent light and can directly focus the coherent/noncoherent light to radiate uniformly on a reasonably defined area, wherein the light source is an array of a plural sets of light emitting devices configured in a matrix of various shape and size, and each set of light emitting device is composed of two paired light emitting elements integrated and aligned in an inclined manner on a Printed Circuit Board (PCB) for controlling the phases and adjusting the discharging angles of the lights emitting from the two light emitting elements.

### 1. FIELD OF THE INVENTION

The present invention relates to an improved cosmetic light therapy system, and more particularly, to an light therapy system having a light source of cosmetic and therapeutic capability that can generate coherent light or noncoherent light and can directly focus the coherent/noncoherent light to radiate uniformly on a reasonably defined area, wherein the light source is an array of a plural sets of light emitting devices configured in a matrix of various shape and size, and each set of light emitting device is composed of two paired light emitting elements integrated and aligned in an inclined manner on a Printed Circuit Board (PCB) for controlling the phases and adjusting the discharging angles of the lights emitting from the two light emitting elements.

### 2. BACKGROUND OF THE INVENTION

As the prevailing of laser technology and the recent discoveries of medical research, many optical devices are developed that can emit light in a continuous mode or in an intense pulsed mode at a specific wave length with a predetermined dwell time and radiate the same on a defined area of a patient for enabling the patient to receive a proper skin treatment, such as increasing radiance, firming skin (i.e. lifting aged and tired skin), improving moisture retention, increasing circulation by increased formation of new capillaries, smoothing skin texture, promoting collagen production which improves elasticity for a more youthful looking skin, increasing oxygenation and restoration of the skins natural cellular activity which keeps skin renewed and fresh, increasing lymphatic system drainage which detoxifies the skin tissue for the healthy look and feel of young skin, and so on.

The state of the art devices suitable for administering cosmetic light therapy can be seen and are disclosed in U.S. Pat. No. 5,549,660, entitles “Method of Treating Acne”, and in U.S. Pat. No. 5,259,380, entitles “Light Therapy System”. As disclosed in the referring U.S. patents, the light therapy system uses a plurality of light emitting diodes (LEDs) mounted either on a planar substrate or a concave spherical substrate as the light source thereof where each of the plural LEDs is independently disposed and each can radiate on a planar zone of circular shape or oval shape. In order to form a comparatively more intense light for a certain skin treatment by coherence stacking or noncoherence stacking the plural independent-transmitted lights emitted from the plural LEDs, the phase σ (P) of the plural LEDs must be adjusted one by one and kept in a stable state after adjusting. It is noted that, to generate the referring coherent light or noncoherent light according to the aforementioned prior arts, it is required to arranged separately the plural LEDs on a circular substrate or a large-sized substrate in a distributed manner and further the plural LEDs are required to be adjusted one by one so as to enable the lights emitting therefrom to maintain a stable phase. However, the circular substrate or the large-sized substrate required for disposing the plural LEDs as seen in the FIG. 3 of the U.S. Pat. No. 5,549,660 (i.e. FIG. 1 of the invention) and the FIG. 2, FIG. 3 of the U.S. Pat. No. 5,259,380 (i.e. FIG. 2 and FIG. 3 of the invention) can not be miniaturized and the same time generate a coherent light or noncoherent light with the same intensity. In another word, the configuration of the LEDs according the referring prior arts set a limit for the size of the substrate required, that is, the smaller the substrate is, the fewer the LEDs can be disposed thereon and thus the lower the light intensity will be generated. Moreover, the phases of the plural LEDs are required to be adjusted one by one and thereafter maintained in a stable state respectively, which will increase the complexity and difficulty on both the fabricating and structural point of view and thus cause the increase of manufacturing cost and is infeasible to be mass produced.

Therefore, it is in great need to have an light therapy system having an improved light source of cosmetic and therapeutic capability that can generate coherent light or noncoherent light and can directly focus the coherent/noncoherent light to radiate uniformly on a reasonably defined area, in one of the plural set of light emitting device forming the light source that two corresponding light emitting elements are integrated and aligned on a PCB directly in an inclined manner for controlling the phases and adjusting the discharging angles of the lights emitting from the two light emitting elements.

### SUMMARY OF THE INVENTION

It is the primary object of the invention to provide an improved cosmetic light therapy system having a light source that can generate coherent light or noncoherent light and can directly focus the coherent/noncoherent light to radiate uniformly on a reasonably defined area without utilizing mirrors, lens or other optical systems.

It is another object of the invention to provide an improved cosmetic light therapy system having a light source that require a comparatively small substrate or PCB for disposing the light emitting elements forming the light source thereof so as to miniaturize the cosmetic light therapy system enabling the system to be lightweight and portable.

Further, another object of the invention is to provide an improved cosmetic light therapy system having a light source that can control the phases and adjust the discharging angles of the lights emitting from the light emitting elements forming the light source while the emitting elements being attached on a substrate or PCB of the improved cosmetic light therapy system such that the complexity of manufacturing the system is reduced and thus the manufacturing cost is lowered.

It is yet another object of the invention is to provide an improved cosmetic light therapy system having a light source that is an array of a plural sets of light emitting devices configured in a matrix of various shape and size enabling the improved cosmetic light therapy system to be used for all kind of light therapy.

To achieve the above description, the present invention provides an improved cosmetic light therapy system having a light source, capable of generating coherent light or noncoherent light and directly focusing the coherent/noncoherent light to radiate uniformly on a reasonably defined area, wherein the light source is an array of a plural sets of light emitting devices configured in a matrix of various shape and size, and each set of light emitting device is composed of two paired light emitting elements integrated and aligned on a PCB in an inclined manner by attaching the same respectively on a pad with inclined surface for controlling the phases and adjusting the discharging angles of the lights emitting from the two light emitting elements, and with respect to various cosmetic and therapeutic requirements, the paired two light emitting elements are a pair selected from the group consisting of two light emitting elements of the same wave length and two light emitting elements of different wave length.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 shows a preferred arrangement of the LEDs of U.S. Pat. No. 5,549,660.

FIG. 2 shows a preferred arrangement of the LEDs mounted on a concave substrate of U.S. Pat. No. 5,259,380.

FIG. 3 shows a preferred arrangement of the LEDs mounted on a planar substrate of U.S. Pat. No. 5,259,380.

FIG. 4 is a schematic illustration showing the structure of a light emitting device composed of two light emitting elements according to a preferred embodiment of the invention.

FIG. 5 is a schematic illustration showing the structure two paired pad of the present invention.

FIG. 6 is a schematic illustration showing two light emitting elements whose discharging angle had been adjusted as are paired and aligned on a substrate according to a preferred embodiment of the invention.

FIG. 7 is a schematic illustration showing a radiating area of two pair light emitting elements according to a preferred embodiment of the invention.

FIG. 8 is a schematic illustration showing a matrix formed by an array of a plural sets of light emitting devices according to a preferred embodiment of the invention.

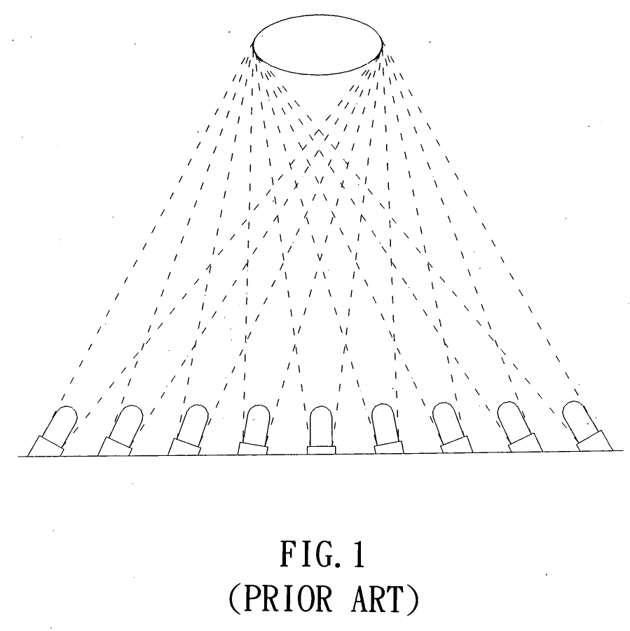

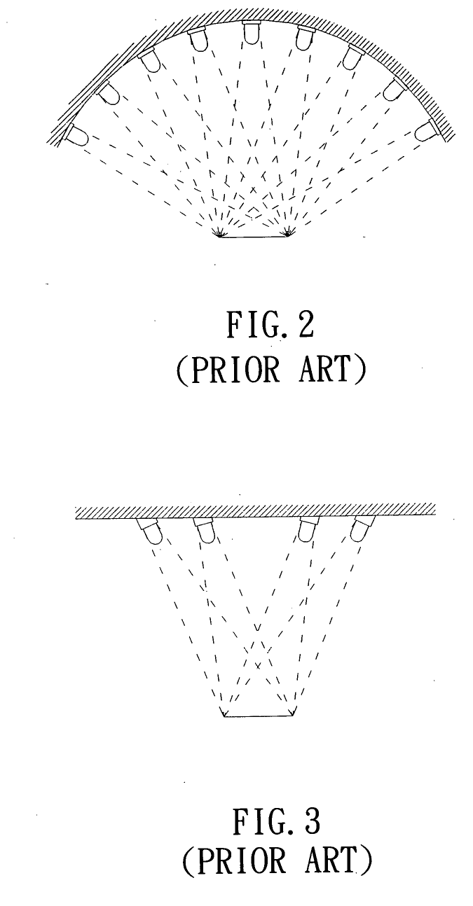

### DESCRIPTION OF THE PREFERRED EMBODIMENT

For your esteemed members of reviewing committee to further understand and recognize the fulfilled functions and structural characteristics of the invention, several preferable embodiments cooperating with detailed description are presented as the follows.

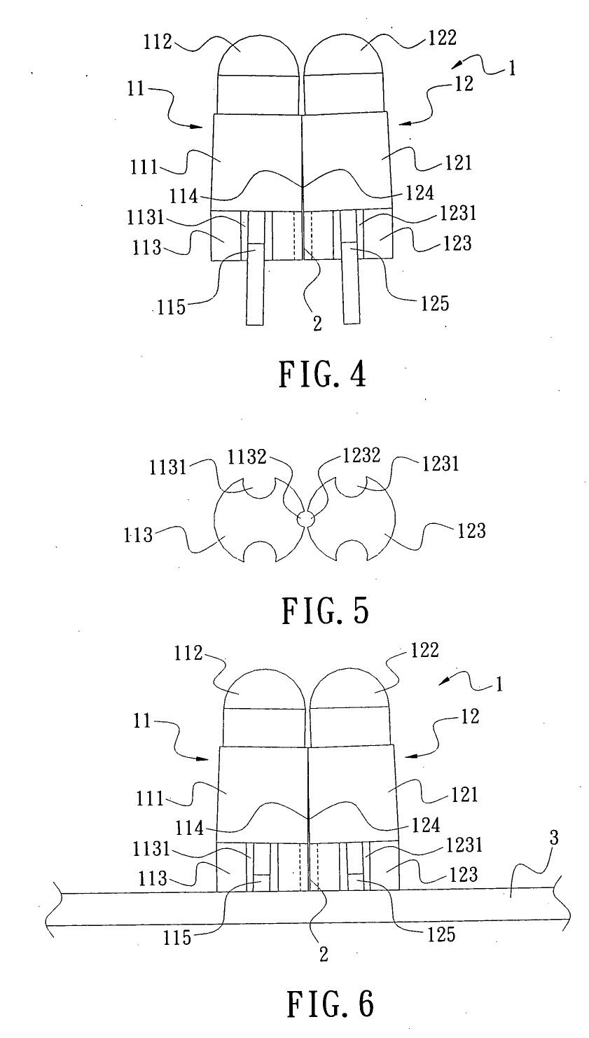

Please refer to FIG. 4, FIG. 5 and FIG. 6, in which a light emitting device 1 composed of two light emitting elements according to a preferred embodiment of the invention is shown. The light source of an improved cosmetic light therapy system of the invention is an array of a plural sets of light emitting devices 1 configured in a matrix of various shape and size, and each set of light emitting device 1 is composed of two paired light emitting elements 11, 12 integrated and aligned on a PCB 3 in an inclined manner by attaching the bodies 111, 121 of the light emitting elements 11, 12 on pads 113, 123 respectively with inclined surface for controlling the phases and adjusting the discharging angles of the lights emitting from the two light emitting elements 11,12, such that the improved cosmetic light therapy system can generate coherent light or noncoherent light and can directly focus the coherent/noncoherent light to radiate uniformly on a reasonably defined area without utilizing mirrors, lens or other optical systems, and thus the improved cosmetic light therapy system can be adapted for a requirement of treating a small area with radiation of high intensity.

As seen in FIG. 4, the discharging angles of the lights emitting from the light emitting elements 11, 12 are readily adjusted while the bodies 111, 112 of the two light emitting elements 11, 12 are attached onto the two pads 113, 123 in respective, since the pads each has an inclined surface and is attached the body of an light emitting element thereby. Further, the two light emitting elements 11, 12 have illuminating zones 112, 122 arranged at the tip thereof respectively. As seen in FIG. 5, the pad 113 has two recesses 1131, 1132 of different diameters while the pad 123 also has two recesses 1231, 1232 of different diameters. As the pins 115, 125 of the light emitting elements 11, 12 is received in the recesses 1131, 1132 while attaching the bodies of the two light emitting elements 11, 12 on the pads 113, 123 in respective, the other two recesses 1132, 1232 can be uses as check marks for aligning the two pair light emitting elements 11, 12 correctly corresponding to each other to be integrated on the PCB 3. Since the inclined surfaces of the pads 113, 123 respectively is designed with a specific inclination angle, the inclined surface of the two pads 113, 123 attaching to the two paired light emitting elements 11, 12 are symmetrically incline to each other enabling the lowest position of an inclined surface of one of the two pads, i.e. the pad 113 for example, is arranged corresponding to the lowest position of that of another pad, i.e. the pad 123, such that an included angle 2 with respect to a specific phase difference is formed naturally between the two corresponding side walls 114, 124 of the two pair light emitting elements 11, 12. As seen in FIG. 6, the included angle 2 is determined and formed as soon as the body of each of the two light emitting element 11, 12 is attached on its pad and the two light emitting element 11, 12 are paired and integrated on the PCB 3, that is, the discharging angles of the lights emitting from the light emitting elements 11, 12 are readily adjusted according to the inclination angles of the inclined surfaces of the pads 113, 123. Therefore, it is noted that the light emitting elements of the present invention do not required to be adjusted one by one that is an improvement over the abovementioned structures of U.S. Patents.

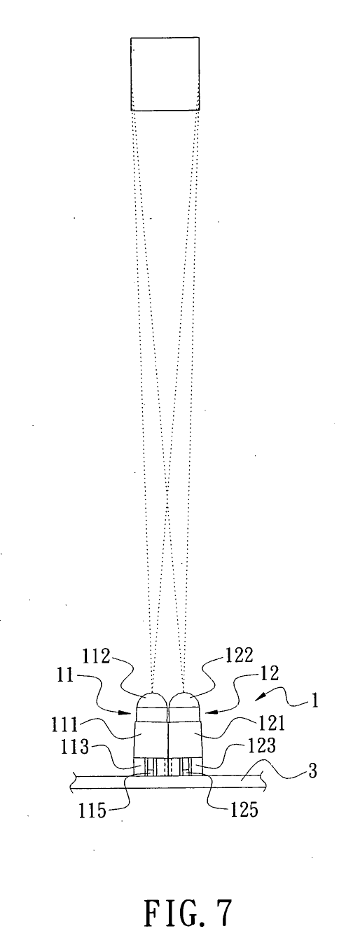

As seen in FIG. 7, by controlling the phases of the lights emitting from the two pair light emitting elements 11, 12 by the inclined surfaces of the pads 113, 123, the improved cosmetic light therapy system can generate coherent light or noncoherent light and can directly focus the coherent/noncoherent light to radiate uniformly on a reasonably defined area without utilizing mirrors, lens or other optical systems, and thus the improved cosmetic light therapy system can be adapted for a requirement of treating a small area with radiation of high intensity.

In a preferred embodiment of the invention, the two paired light emitting elements 11, 12 respectively is an element selected from the group consisting of an Light Emitting Diode (LED), a Resonant Cavity Light Emitting Diode (RCLED), a LASER, a Vertical Cavity Surface Emitting Laser (VCSEL). Moreover, the lights discharging from the two paired light emitting elements 11, 12 can be lights of the same wave length or lights of different wave lengths, that the selection of wavelength and the combination thereof are based on the treatment requirement. For instance, green light laser of 530 nm wavelength and blue light laser of 410 wavelength for treating vascular lesions or red blemishes on the skin; red light laser of 630 wavelength for cosmetic procedures such as removing tattoos, smoothing skin texture, and promoting collagen production; yellow light laser of 590 wavelength for treating vascular lesions, skin treatment and photodynamic therapy (PDT).

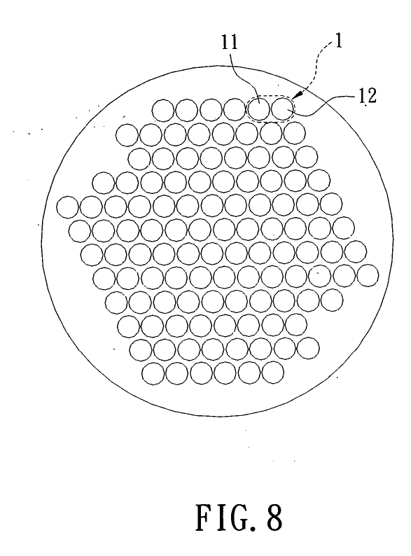

As seen in FIG. 8, the light source of the improved cosmetic light therapy system is an array of a plural sets of light emitting devices 1 configured in a matrix of various shape and size enabling the improved cosmetic light therapy system to be used for all kind of light therapy, wherein each set of light emitting device 1 is composed of two paired light emitting elements 11, 12, so that the combination of the plural sets of light emitting devices 1 can generate a comparatively more intense coherent light or noncoherent light and can directly focus the coherent/noncoherent light to radiate uniformly on a reasonably defined area.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. An improved cosmetic light therapy system, having a light source composed of an array of a plural sets of light emitting devices, each set of light emitting device being composed of two paired light emitting elements integrated and aligned on a PCB in an inclined manner by attaching the same respectively on a pad with inclined surface, wherein an included angle with respect to a specific phase difference is formed between the two paired light emitting elements for enabling the lights discharging respectively therefrom to be a stacked light selected from the group consisting of a coherent light and a noncoherent light.

2. The improved cosmetic light therapy system of claim 1, wherein the inclined surface of the pad has a specific inclination angle.

3. The improved cosmetic light therapy system of claim 1, wherein the inclined surface of the two pads attaching to the two paired light emitting elements are symmetrically incline to each other enabling the lowest position of an inclined surface of one of the two pads is arranged corresponding to the lowest position of that of another pad.

4. The improved cosmetic light therapy system of claim 2, wherein the specific inclination angles of the inclined surfaces of the pads of the two paired light emitting elements control and determine the phase difference between the lights discharging respectively from the two paired light emitting elements, and the discharging angles of the lights discharging respectively from the two paired light emitting elements are completely adjusted while the two paired light emitting elements being attached on the pads in respective.

5. The improved cosmetic light therapy system of claim 1, wherein the light emitting element is an element selected from the group consisting of an Light Emitting Diode (LED), a Resonant Cavity Light Emitting Diode (RCLED), a LASER, a Vertical Cavity Surface Emitting Laser (VCSEL).

6. The improved cosmetic light therapy system of claim 1, wherein the lights discharging from the two paired light emitting elements are lights of the same wave length.

7. The improved cosmetic light therapy system of claim 1, wherein the lights discharging from the two paired light emitting elements are lights of different wave lengths.

8. The improved cosmetic light therapy system of claim 1, wherein the light source is an array of a plural sets of light emitting devices configured in a matrix of various shape and size.
